SEQUENCE LISTING (26 sequences)

SEQ ID NO: 1 - DNA, 1650 bp, Bacillus pumilus
  CDS (1)..(1650)
atgaatcaattaattgaaaatgttaatctacaaaaattaaggggtgggtattacacccctaaagttattgctgactttttatgtcaatggagtattcaagatgacacaaagagtgtacttgaacccagttgtggagatggtaattttattgaatcggcaatacttaggttcaaagaacttagtatagataatgaacaacttaaaggaagaattacaggagtagagctaattgaagaagaagctttgaaagttcaaaatcgagcaaatgagttgggggttgataaaaactcaatagtaaatagtgacttctttcaatttgtaaaagataataagaataaaaaatttgatactattattggtaatccaccattcataagataccaaaactttcctgaagagcatcgtagtatagccatggaaatgatggaggaactaggtttaaaacctaataaacttacaaatatctgggttccatttctagtggtatctgctacattacttaatgaacaaggaaagatggctatggttataccggctgaattatttcaggtaaagtatgcagcagaaacaagaatttttttatcaaagtttttcgatcgtatcactataattacatttgaaaaacttgtttttgaaaatatccaacaggaagttatactacttctttgtgaaaagaaagttaataaaggtaaaggaattcgggttattgaatgcgagaacttagatggattaaattccattgattttgtagctataaatggttcaaatgttaaacctattgaacaccgtactgaaaagtggacaaagtatttcttaaacgaagatgaaatacttcttttacagagtttaaaggaagacaaacgcgttaaaaattgtaatgactattttaagacagaagttggcttagttactggacgaaacgaattctttatgatgaaagaaaaccaagtaaaagaatggaatctagaagaatatacaatacctgttacaggtaggtccaatcagttaaaaggtataacatttacagaaaatgattttcatgaaaattcaatggaacaaaaggcaattcacctatttttgccaccagatgaagattttgaaaagttaccgattgagtgtcaaaattatatcaagtatggggaagaaaaaggcttccatcaaggctataaaaccagaattagaaaacgttggtatataactccatctagatgggttccagatgcttttgctttaagacaggttgatggctatccaaaactaattttaaatgaaaccgacgcttcttctactgatacaattcatagggttagatttaaagaaggtataaatgaaaagttagccgtagtttcatttttgaactcactcacttttgcatcttcagaaataacggggagaagttatggtggtggtgttatgacattcgaaccaactgaaattggagaaatcctaataccttcctttgataacttatccattgattttgataaaattgatgccttaattcgagaaaaggagattgaaaaagtccttgatattgttgatgaagctttacttataaaatatcatgggtttagtgagaaagaagtaaaacagcttcgagggatatggaagaaactttctcagagaagaaacaatagaacgaagaaataa

SEQ ID NO: 2 - protein, 549 aa, Bacillus pumilus
MNQLIENVNLQKLRGGYYTPKVIADFLCQWSIQDDTKSVLEPSCGDGNFIESAILRFKELSIDNEQLKGRITGVELIEEEALKVQNRANELGVDKNSIVNSDFFQFVKDNKNKKFDTIIGNPPFIRYQNFPEEHRSIAMEMMEELGLKPNKLTNIWVPFLVVSATLLNEQGKMAMVIPAELFQVKYAAETRIFLSKFFDRITIITFEKLVFENIQQEVILLLCEKKVNKGKGIRVIECENLDGLNSIDFVAINGSNVKPIEHRTEKWTKYFLNEDEILLLQSLKEDKRVKNCNDYFKTEVGLVTGRNEFFMMKENQVKEWNLEEYTIPVTGRSNQLKGITFTENDFHENSMEQKAIHLFLPPDEDFEKLPIECQNYIKYGEEKGFHQGYKTRIRKRWYITPSRWVPDAFALRQVDGYPKLILNETDASSTDTIHRVRFKEGINEKLAVVSFLNSLTFASSEITGRSYGGGVMTFEPTEIGEILIPSFDNLSIDFDKIDALIREKEIEKVLDIVDEALLIKYHGFSEKEVKQLRGIWKKLSQRRNNRTKK

SEQ ID NO: 3 - DNA, 3030 bp, Bacillus pumilus
  CDS (1)..(3030)
atgcatataagtgagttagtagataaatacaaagcgcatagaagtacttttttaaaaccaacttataatgaaactcaactaaggaatgattttatagacccacttctaaaatctttaggatgggatgttgataataccaaaggaaaaacacatattctaagagatgtcattcaagaagaatacatagaaataaaagatgaggagacaaagaaaaatccagattatacacttcgtataaacggtacgagaaagctgtttgtagaggttaagaaaccgtcttttaatattttgaaatcagctaaagcagccttccaaacaagaagatatggttggagtgctaaccttggtatttcagtacttacaaatttcgagcatctagttatttatgattgtagatatacgcctgacaaatccgacaatgaacatattgctagatataaagttttctcttacgaggaatatgaagaagcatttgatgaaataaaggatataatttcatatgagtcagccaactcaggtgctctggacgaaatgtttgatgtaaatacaagagttggtgaaacgtttgacgagtattttttacagcaaattgagaattggcgcgaaaagctagctaaaactgcaattaaaaataacaccgaattaggtgaagaggacgtcaattttattgtccaaagactattaaacagaattatttttcttagagtttgtgaagatagaaccattgaaaaatatgaaacaattaaaagtataaaaaactatgaggaattaaaagatctgtttcaaaagtctgataggaaatttaattcaggtctctttgacttcatagatgatacgctcttgcttgaggttgaaattgattcgaatgtattgatagaaatttttagtgatttatatttcccacaaagcccatatgatttttctgttgtcgatccaacaatattaagccagatatatgaacgttttctaggtcaagaaataattatagagtcaggtggtacatttcacattacggagtcaccagaagttgcggcgtccaatggtgttgttccaactccaaaaattatcgtcgaacagatagtgaaagacactttaacgccccttacggaaggcaaaaaatttaatgagctatgtaacttaaaaatagcagatatatgttgtggatcaggaactttcctaatttcaagttatgactttctagtagagaaagtaatggaaaagataatagaagagaacatcgatgattcagatttagtatatgaaactgaagaagggctaattttgacacttaaagcaaaaagaaatatcttggagaataatttgtttggtgttgatgttaatccatacgctgttgaagtagctgagttcagtttattattaaagctattagaaggtgagaatgaggcatcggttaataatttcattcacgagcatgaggataaaatattaccggatttaacatctattattaaatgtggaaacagcttagtagataataagttttttgaattcatgccagaatcgttagaggacgatgaaatcttatttaaggctaatccatttgaatgggaagaggagtttccagatattatggcaaatggtggctttgatgctattataggaaatccaccttatgttcgaatacagaacatgaaaaaatatagtcctgaggaaattgaatattatcaatcaaaagactctgaatatactgttgcaaaaaaagaaacagttgacaagtattttttatttattgagagagcattaatattactcaatcctactgggctgttgggttatataataccgcataaattctttattacaaaaggtggtaaggaactaagaaagttcatagctgaaaaacatcaaatatcaaaaattataaattttggtgttacacaggtctttccaggaagagcgacatatacggctattttaattatccaagcaaataaaatggcacagttcaagtataagaaagtaagtaatatatcagcagaaaccctagattctgaagaaaatacgtgtgtttatagctcagaaaagtataattctgacccttggatatttttatctcctgaaacagaagctgtttttactaaatttacagaagctcaatttgagaaacttggagaaatcactgatataagtgtaggactacaaacaagcgctgataaaatatatatttttattcctgaaaatgaaacttcagatacatatatatttaattataaagggaaaagatatgaaatagaaaaatctatatgttgcccagctatctatgacttatcttttggttcttttgaaagcattcagggaaatgcacaaatgatattcccttatgaaatcagagatgaagaagcatatctactagaggaagaaacgcttgaaaatgattatcctcttgcttggaattatttgaatgagtttaaagaagctcttgaaaaaagaagcttacaaggccgtaatccgaaatggtatcaatatggtcggtcccaaagtttatcaaaatttcatgataaagaaaaactgatatggaccgtacttgctacgaaacccccgtatgtacttgataggaataacctgttatttactggtggtggaaacggaccgtattatggtttaattaaccaatctatttactctttgcattattttttaggtattctttcacatcctgtaatagaaagtatggtaaaagcaagggccagtgaatttaggggatcatattattctcatggaaaacaatttattgagaaaatcccaattagaaagattgattttgatgatcaagatgaggtagacaaatataatacggtggtcacaacagtagaaaaattaattataactaccgatagaattaaaagtgagagcaatggaccccggaggagaatgttaagaagaaggttagatgctttgtctaatcaacttatccaggttattaatgaactttataatatcagtgacgaagaatatacgacagttttgaatgatgaaatgttgacagcggcgttaggagaagaaaaatga

SEQ ID NO: 4 - protein, 1009 aa, Bacillus pumilus
MHISELVDKYKAHRSTFLKPTYNETQLRNDFIDPLLKSLGWDVDNTKGKTHILRDVIQEEYIEIKDEETKKNPDYTLRINGTRKLFVEVKKPSFNILKSAKAAFQTRRYGWSANLGISVLTNFEHLVIYDCRYTPDKSDNEHIARYKVFSYEEYEEAFDEIKDIISYESANSGALDEMFDVNTRVGETFDEYFLQQIENWREKLAKTAIKNNTELGEEDVNFIVQRLLNRIIFLRVCEDRTIEKYETIKSIKNYEELKDLFQKSDRKFNSGLFDFIDDTLLLEVEIDSNVLIEIFSDLYFPQSPYDFSVVDPTILSQIYERFLGQEIIIESGGTFHITESPEVAASNGVVPTPKIIVEQIVKDTLTPLTEGKKFNELCNLKIADICCGSGTFLISSYDFLVEKVMEKIIEENIDDSDLVYETEEGLILTLKAKRNILENNLFGVDVNPYAVEVAEFSLLLKLLEGENEASVNNFIHEHEDKILPDLTSIIKCGNSLVDNKFFEFMPESLEDDEILFKANPFEWEEEFPDIMANGGFDAIIGNPPYVRIQNMKKYSPEEIEYYQSKDSEYTVAKKETVDKYFLFIERALILLNPTGLLGYIIPHKFFITKGGKELRKFIAEKHQISKIINFGVTQVFPGRATYTAILIIQANKMAQFKYKKVSNISAETLDSEENTCVYSSEKYNSDPWIFLSPETEAVFTKFTEAQFEKLGEITDISVGLQTSADKIYIFIPENETSDTYIFNYKGKRYEIEKSICCPAIYDLSFGSFESIQGNAQMIFPYEIRDEEAYLLEEETLENDYPLAWNYLNEFKEALEKRSLQGRNPKWYQYGRSQSLSKFHDKEKLIWTVLATKPPYVLDRNNLLFTGGGNGPYYGLINQSIYSLHYFLGILSHPVIESMVKARASEFRGSYYSHGKQFIEKIPIRKIDFDDQDEVDKYNTVVTTVEKLIITTDRIKSESNGPRRRMLRRRLDALSNQLIQVINELYNISDEEYTTVLNDEMLTAALGEEK

SEQ ID NO: 5 - DNA, 24 bp, Bacillus pumilus
gtggaaacggaccgtattatggtt

SEQ ID NO: 6 - DNA, 24 bp, Bacillus pumilus
caccagtaaataacaggttattcc

SEQ ID NO: 7 - DNA, 27 bp, Bacillus pumilus
ttcgtagcaagtacggtccatatcagt

SEQ ID NO: 8 - DNA, 27 bp, Bacillus pumilus
ccgtatgtacttgataggaataacctg

SEQ ID NO: 9 - DNA, 24 bp, Bacillus pumilus
aggaactaagaaagttcatagctg

SEQ ID NO: 10 - DNA, 24 bp, Bacillus pumilus
atgcggtattatataacccaacag

SEQ ID NO: 11 - DNA, 24 bp, Bacillus pumilus
tgacgtcctcttcacctaattcgg

SEQ ID NO: 12 - DNA, 24 bp, Bacillus pumilus
gagtttgtgaagatagaaccattg

SEQ ID NO: 13 - DNA, 48 bp, Bacillus pumilus
agcggatccggaggtaaataaatgaatcaattaattgaaaatgttaat

SEQ ID NO: 14 - DNA, 42 bp, Bacillus pumilus
aagggggcatgcttatacttatttcttcgttctattgtttct

SEQ ID NO: 15 - DNA, 51 bp, Bacillus pumilus
caaggatccggaggtaaataaatgcatataagtgagttagtagataaatac

SEQ ID NO: 16 - DNA, 36 bp, Bacillus pumilus
ttaggatcctcatttttcttctcctaacgccgctgt

SEQ ID NO: 17 - DNA, 54 bp, Bacillus pumilus
caccaatctagaggaggtaaataaatgcatataagtgagttagtagataaatac

SEQ ID NO: 18 - DNA, 42 bp, Bacillus pumilus
tgaaatctcgagttatcctgatccacaacatatatctgctat

SEQ ID NO: 19 - DNA, 54 bp, unknown; identical to SEQ ID NO: 17
  Synthetic primer

SEQ ID NO: 20 - DNA, 39 bp, unknown
  Synthetic primer
gtttatacgaagtgtataagctggatttttctttgtctc

SEQ ID NO: 21 - DNA, 39 bp, unknown
  Synthetic primer
gagacaaagaaaaatccagcttatacacttcgtataaac

SEQ ID NO: 22 - DNA, 36 bp, unknown; identical to SEQ ID NO: 16
  Synthetic primer

SEQ ID NO: 23 - DNA, 54 bp, unknown
  Synthetic primer
ggtggttctagaggaggtaaataaatgtctaatgaaaattataacattgatttc

SEQ ID NO: 24 - DNA, 39 bp, unknown
  Synthetic primer
ggtggtgagctcctattgacataatcgatcatcaagaag

SEQ ID NO: 25 - DNA, 42 bp, unknown
  Synthetic primer
atagggtggattgcctaatattacatcaaagccaccatttgc

SEQ ID NO: 26 - DNA, 40 bp, unknown
  Synthetic primer
tttgatgtaatattaggcaatccaccctatataagaattc

(19) United States
(12) Patent Application Publication
(10) Pub. No.: US 2004/0219584 A1
(43) Pub. Date: Nov. 4, 2004

(54) Methods for altering the cleavage specificity of a type IIG restriction endonuclease

(76) Inventors: Shuang-yong Xu, Lexington, MA (US); Daniela Kobbe, Karlsruhe (DE); Zhenyu Zhu, Beverly, MA (US); James Samuelson, Danvers, MA (US)

Correspondence Address: NEW ENGLAND BIOLABS, INC., 32 TOZER ROAD, BEVERLY, MA, 01915, US

(21) Appl. No.: 10/800,946

(22) Filed: Mar. 15, 2004

Related U.S. Application Data
(63) Continuation-in-part of application No. 10/150,028, filed on May 17, 2002, now abandoned.
Application No. 10/150,028 is a division of application No. 09/693,146, filed on Oct. 20, 2000, now Pat. No. 6,413,758.

(51) Int. Cl. C12Q 1/68; C07H 21/04; C12N 9/22; C12N 15/85

(52) U.S. Cl. 435/6; 435/69.1; 435/455; 435/199; 435/320.1; 435/325; 536/23.2

(57) ABSTRACT

Methods are provided for altering the cleavage specificity of a Type IIG restriction endonuclease, the Type IIG restriction endonuclease being characterized by a cleavage domain adjacent to a methylase domain, the methylase domain located adjacent to a specificity domain. The method includes ligating DNA or protein sequences to form a fusion DNA or fusion protein. Where a fusion DNA is formed, the host cell is transformed with the fusion DNA to express a Type IIG restriction endonuclease with altered cleavage specificity.

### CROSS REFERENCE

This application is a continuation-in-part of U.S. patent application Ser. No. 10/150,028 filed May 17, 2002, which is a divisional application of U.S. application Ser. No. 09/693,146 filed Oct. 20, 2000, now U.S. Pat. No. 6,413,758 issued Jul. 2, 2002, each of which is hereby incorporated by reference in its entirety.

### BACKGROUND OF THE INVENTION

Type II restriction endonucleases are a class of enzymes that occur naturally in bacteria and in some viruses. When they are purified away from other bacterial proteins, restriction endonucleases can be used in the laboratory to cleave DNA molecules into small fragments for molecular cloning and gene characterization.

Restriction endonucleases act by recognizing and binding to particular sequences of nucleotides (the ‘recognition sequence’) along the DNA molecule. Once bound, they cleave the molecule within, to one side of, or to both sides of the recognition sequence. Different restriction endonucleases have affinity for different recognition sequences.

Bacteria that produce restriction endonucleases, protect their own DNA by methylating nucleotides at the cleavage site of the endonuclease. The coordinated production of a restriction endonuclease and a specific methylase is called a restriction-modification (R-M) system.

Methyltransferases are complementary to restriction endonucleases and they provide the means by which bacteria are able to protect their own DNA and distinguish it from foreign, infecting DNA. Modification methylases recognize and bind to the same recognition sequence as the corresponding restriction endonuclease, but instead of cleaving the DNA, they chemically modify one particular nucleotide within the sequence by the addition of a methyl group (C5-methyl cytosine, N4-methyl cytosine, or N6-methyl adenine). Following methylation, the recognition sequence is no longer cleaved by the cognate restriction endonuclease. The DNA of a bacterial cell is always fully modified by the activity of its modification methylase. It is therefore completely insensitive to the presence of the endogenous restriction endonuclease.

By means of recombinant DNA technology, it is now possible to clone genes and overproduce the enzymes in large quantities. Restriction endonucleases have highly specific recognition and cleavage sites. It would be desirable to expand the repertoire of restriction endonucleases by retaining a particular cleavage position while modifying the recognition site of an enzyme.

### SUMMARY OF THE INVENTION

In a preferred embodiment, a method is provided for altering the cleavage specificity of a Type IIG restriction endonuclease where the Type IIG restriction endonuclease is characterized by a cleavage domain adjacent to a methylase domain, the methylase domain located adjacent to a specificity domain. The method includes ligating a first DNA sequence and a second DNA sequence to form a fusion DNA. The first DNA sequence includes a DNA segment encoding a catalytic domain and an N-terminal portion of a methylase domain of a first Type IIG restriction endonuclease. The second DNA sequence includes a DNA segment encoding a specificity domain and a C-terminal portion of a methylase domain of a second Type IIG restriction endonuclease, such that the ligation occurs between sequences encoding the methylase domain. A preparation of host cells are then transformed with the fusion DNA for expressing a Type IIG restriction endonuclease with altered cleavage specificity.

In an additional embodiment, the method described above further includes introducing a mutation into the cleavage domain to enhance the viability of the transformed host cell.

In an additional embodiment, a method described above further includes a sequence corresponding to the N-terminal portion of the methylase which terminates within a methylase conserved motif selected from motifs X, I, II, III, IV, V, VI, VII and VIII.

In an additional embodiment, a method described above further includes a sequence corresponding to the C-terminal portion of the methylase which terminates in a methylase conserved motif selected from motifs X, I, II, III, IV, V, VI, VII and VIII. In preferred embodiments, the N-terminal portion and the C-terminal portion of the methylase are non-overlapping.

In a particular example of the above, the sequence corresponding to the N-terminal portion of the methylase motif terminates between the amino acid sequence encoding motif III and amino acids-NPPY in motif IV.

In one embodiment, ligation occurs by means of a linker sequence attached to the N-terminal portion of the methylase domain and the C-terminal portion of the methylase domain on the first and second DNA segment.

In an embodiment, the fusion DNA encodes an active methylase domain.

In an embodiment, the first and second Type IIG endonucleases are endonucleases with defined cleavage and recognition sites or alternatively, the first Type IIG endonuclease is an endonuclease with defined cleavage and recognition sites and the second Type IIG endonuclease is characterized by a bioinformatic search of a microbial sequence database.

In one embodiment of the invention, a method is provided for forming a non-natural, functional Type IIG restriction endonuclease, wherein the Type IIG restriction endonuclease is characterized by a functional cleavage domain, a functional methylase domain and an altered functional specificity domain compared with a natural form of the functional Type IIG endonuclease. The method includes (a) inserting into a DNA encoding the methylase domain or the specificity domain of the natural form of the functional Type IIG endonuclease, a mutation or a nucleic acid linker sequence for inactivating optionally the cleavage domain; and inactivating (i) the functional methylase domain and the specificity domain or (ii) the functional methylase domain or the functional specificity domain; (b) ligating to the DNA at the mutation or at the linker, a DNA encoding (i) a portion of the methylase and specificity domain or (ii) a portion of the methylase or specificity domain to form a fusion DNA; and (c) transforming a host cell having a marker for detecting expression of a colony expressing a non-natural functional Type IIG restriction endonuclease.

For example, in the above method, the mutation may be positioned within a conserved motif in the methylase domain or the mutation may be a deletion at a 5′-end of the DNA encoding the specificity domain or the mutation is a deletion within the specificity domain. Alternatively, where a linker is utilized, the linker may be a transposon mediated linker insertion sequence. The linker may contain a restriction endonuclease cleavage site which is unique within the DNA encoding the restriction endonuclease.

### BRIEF DESCRIPTION OF THE DRAWINGS

FIG. 1 shows a gene organization of BpmI restriction-modification system. Genes BpmIRM and BpmIM1 code for BpmI endonuclease (BpmI endonuclease-methylase fusion protein and BpmI M1, respectively. BpmI-Δ#1, BpmI-Δ#2, and BpmI-Δ#3 are deletion mutants with deletions in the methylation or specificity domains.

FIG. 2 shows a DNA sequence of BpmIM1 methylase gene (BpmIM1) (SEQ ID NO:1) and its encoded amino acid sequence (SEQ ID NO:2).

FIG. 3 shows a DNA sequence of BpmI endonuclease gene (BpmIRM) (SEQ ID NO:3) and its encoded amino acid sequence (SEQ ID NO:4).

FIG. 4 shows a recombinant BpmI endonuclease activity in column fractions following heparin Sepharose chromatography (Amersham Biosciences, Piscataway, N.J.). Lane 1: purified native BpmI endonuclease; lanes 2 to 23: heparin Sepharose column fractions. Fractions 11 to 14 gave rise to complete BpmI digestion of λ DNA. The remaining fractions contain no or partial BpmI activity. Lane 24: 1 kb DNA size marker.

FIG. 5 shows a functional domain organization of BpmI R-M fusion protein. The conserved amino acid motifs I and IV are indicated. The linear order of the conserved aa motifs in the γ-type methylase is motifs X-I-II-III-IV-V-VI-VII-VIII. The specificity domain is shared between the endonuclease and methylase activites. However, some amino acid residues may play roles in both specific binding (specificity determinant) and catalysis.

FIG. 6 shows a cleavage-deficient and methylation-proficient BpmI variants. Lane 1, protein size marker; lane 2, uncut plasmid DNA (pET28-BpmIRM); lanes 3-20, BpmI variants. Lanes 3, 5-9, 12-14, 17, 20, R⁻M⁺ variants. Note, the R-M fusion methylase (M1) only conferred partial resistance to the plasmid. Full resistance requires a second methylase, M.BpmI (M2).

FIG. 7 shows AcuI deletion variant D80A/D(520-1000) in SDS-PAGE gel in the presence and absence of IPTG. M, protein size marker (in kDa); lanes 1, 3, 5, 7, 9, 11, IPTG-induced cell extracts; lanes 2, 4, 6, 8, 10, 12, non-induced cell extracts. The predicted molecular weight of the deletion variant is 59 kDa.

FIG. 8 shows in vivo SOS induction assay (endo-blue assay) for the BpmI/BsgI chimeric enzyme on X-gal plates. Blue colony transformants were re-streaked on X-gal plates to test the stability of blue phenotype. First line streak, “\” corresponds to low activity BstYI mutants (blue colonies, positive control); second line streak, “-” corresponds to white colonies, vector control; third, fourth, and fifth streak lines, blue colonies of BpmI/BsgI chimeric clones. Sixteen clones remained blue while eight clones turn white or partial blue.

### DETAILED DESCRIPTION OF THE EMBODIMENTS

The methods described herein address the problem of how to modify the specificity of any restriction endonuclease by genetic manipulation. A chimeric endonuclease is formed in which the specificity domain of a particular Type IIG restriction endonuclease is altered by substitution of part or all of the specificity and/or methylase domains with a complementary portion of the specificity and methylase domains from a second Type IIG restriction endonuclease or a methylase. The complementary portion may be selected from any Type IIG restriction endonuclease or a methylase identified in REBASE or identified by bioinformatic techniques described in U.S. Pat. No. 6,383,770.

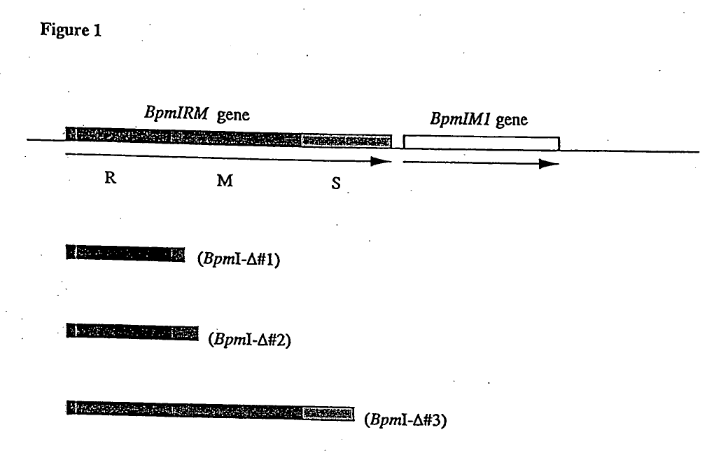

The molecular architecture of Type IIG enzymes is composed of three functional domains, the catalytic, methylase, and specificity domains (R⁺-M⁺-S⁺) (FIG. 1). These domains are generally aligned from the N-terminal end to the C-terminal end in the order of catalytic domain, methylase domain and specificity domain. Whereas the methylase domain contains highly conserved regions (nine motifs have been identified), the catalytic domain and the specificity domain are generally highly variable between different Type IIG restriction endonucleases. Examples of Type IIG enzymes with established recognition and cleavage sites include BpmI, AcuI and BsgI.

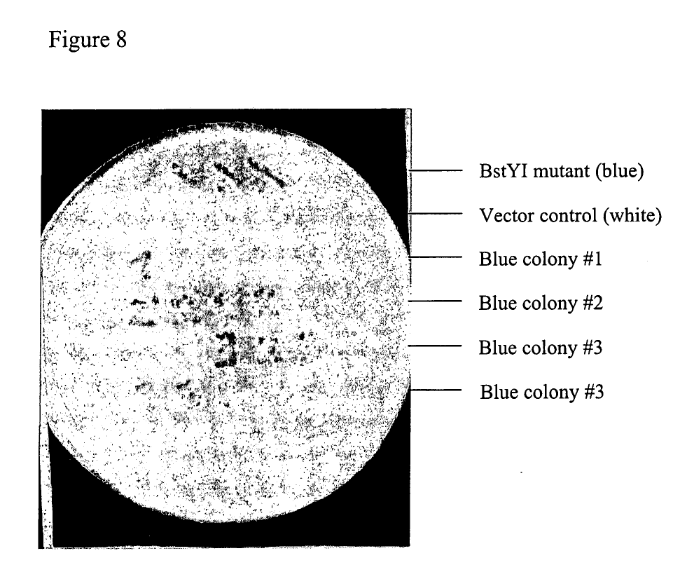

Particular examples are provided below to demonstrate how functional chimeric endonucleases can be formed having altered specificity. The methods described are not intended to be limited to these examples but rather are applicable to any Type IIG restriction endonuclease characterized by a single specificity domain for both a methylase and catalytic domain. Accordingly, alteration of the specificity domain or switching specificity domains between different Type IIG restriction endonucleases or between a Type IIG endonuclease and a methylase results in alteration of the specificity domain of both the methylase and the restriction endonuclease domain in the target Type IIG endonuclease. In vivo assays to determine whether a functionally active restriction endonuclease with altered specificity has been successfully produced is described in Example 8 using an E. coli strain carrying the dinD:IacZ fusion (see U.S. Pat. No. 5,498,535 herein incorporated by reference). This assay permits 10,000 colonies to be screened on one plate with the positive colonies appearing blue and the negative colonies, white (FIG. 8). This in vivo assay avoids time-consuming analysis of individual transformed colonies.

The alteration of specificity of a Type IIG restriction endonucleases described herein results from any of two distinct approaches.

### Linker Insertion

In one approach to the above problem, a nucleic acid linker is inserted into DNA encoding the methylase or specificity domain of one Type IIG restriction endonuclease. The linker may be sufficient in length to encode 3-12 amino acids. A DNA encoding a complementary portion of a second Type IIG restriction endonuclease or a portion of a complementary region of a second Type IIG restriction endonuclease or all or part of an independent y-type methylase (not derived from a Type IIG endonuclease) but containing a specificity region which is ligated to the linker. The chimeric DNA encodes a functional restriction endonuclease with altered specificity. Example 3 describes how a DNA linker coding for up to about 10 amino acids may be inserted between the coding region for a methylase and the coding region for the restriction endonuclease (in this example, BpmI) such that a second methylase region and a specificity region is added to the linker.

It will be clear to one of ordinary skill in the art, that any restriction endonuclease cleavage site that occurs only once in the DNA encoding the Type IIG endonuclease may be used as an insertion site for a linker and consequently for adding all or part of a complementary methylase/specificity domain. Examples of single cut sites in the BpmIRM gene are AfeI, BspHI, BclI, HindIII, and PacI. The linker insertion may inactivate the endonuclease catalytic activity (R⁻M⁺) or the methylase activity (R⁺M⁻) or both activities (R⁺M⁻) However, subsequent to ligation, the in vivo assay described in Example 8 can rapidly distinguish active from inactive transformed colonies.

One approach to facilitate linker insertion uses a drug resistant cassette flanked by convenient restriction sites. Following the introduction of the drug resistant cassette (for example, selection of Km^{R} colonies), the majority of the cassette is removed by restriction digestion and religation, leaving only 3 to 12 codons in-frame insertions in the gene.

Alternative to a random single cutting site or a specific cutting site for insertion of a linker, linkers may be randomly inserted using a transposon-mediated linker insertion system (GPS™-LS linker scanning system, New England Biolabs, Inc., Beverly, Mass.). This system generates insertion of, for example, 15 bp “linkers” (5 amino acids) at random positions throughout the R-M-S gene. The linker scanning mutagenesis is carried out by introduction of a transposon carrying a drug resistance. Following transposon insertion into the target gene carried on a plasmid and drug resistant selection, the majority of the transposon is removed by restriction digestion and ligation. Religation results in a 15 bp insertion (5 amino acids). Protein segments that are tolerant to linker insertion can be identified this way and a DNA segment encoding a novel binding specificity can be inserted afterwards in a manner similar to that described above. Again the in vivo assay described in Example 8 is a rapid method for screening thousands of colonies for endonuclease activity. Positive colonies include colonies having R⁺M⁻S⁺ or R⁻M*S⁺ DNA (M*indicates under methylation).

### Non-linker Dependent Techniques

In an approach exemplified in Examples 5-8, mutations in one of the conserved motifs of the methylase domain of the target Type IIG restriction endonuclease are created which act as a site for exchange of DNA encoding the specificity domains and a part or all of the complementary portion of the methylase. These mutations can be introduced by PCR mutagenesis described in Example 6 but mutations may also be introduced according to other methods know to one of ordinary skill in the art. For example, U.S. patent application, Ser. No. 10/208,557 herein incorporated by reference describes an alternate method for creating mutations at target sites in a DNA. In Example 6, we show the construction of an AcuI deletion variant carrying deletion in the methylase domain and specificity domain for use in formation of chimeric proteins by exchange of domains with a second Type IIG restriction endonuclease or y-type methylase.

Example 7 describes how naturally occurring mutations can be utilized to generate a restriction endonuclease with altered specificity. In this example 6, ThaIVp is used for forming a chimeric enzyme with altered specificity. In Example 8, a chimera between BpmI and BsgI is described.

An alternative approach to the above non-linker techniques is to construct a restriction endonuclease variant library containing nested C-terminal amino acid deletions. The deletion can be carried out by, for example, nuclease digestion or Bal31 nuclease digestion from the C-terminal coding end. After nuclease treatment, the ends are filled-in by Klenow fragment and then ligated to a new DNA specificity (binding) domain. The library DNA carrying new DNA specificity domain is then screened by DNA binding assays or by in vivo SOS-induction assay, or other functional assays.

In any of the above approaches either utilizing linkers or relying on mutations, ligation of DNA segments from different sources can be achieved using any of the established techniques in the art such as ligase mediated ligation, ligation using single stranded ends. (see U.S. Pat. No. 6,660,475, US 2003-0194736 A1). In addition, ligation of proteins or peptides may be achieved using the intein mediated techniques described in U.S. Pat. No. 5,496,714 WO 00/18881 and WO 00/47751.

If the resulting chimeric protein that is produced form cells transformed with ligated chimeric DNA is insoluble due to aberrant folding and forms inclusion body, the protein can be refolded using various denaturing agents and refolded by slow dialysis into suitable buffer conditions.

All references cited herein are incorporated by reference.

### EXAMPLES

### Example 1

### Techniques

PCR, site-directed mutagenesis PCR procedure: PCR conditions are of 94° C. for 30 sec, 55° C. for 30 sec, 72° C. for 30 sec to 3 min for 13-25 cycles with 2 to 4 units of Vent® DNA polymerase in the presence of 2-10 mM MgCl2, dNTP, DNA template, and 1x Thermopol buffer. In some cases, the PCR products were purified from a low-melting agarose gel and treated with b-agarase. The DNA was precipitated with ethanol and salt. After vacuum drying, the DNA was resuspended in TE buffer and used for template for assembling PCR or for restriction digestion.

Plasmid DNA preparation procedure: Qiagen spin columns were used to prepare plasmid DNA. Cells lysis, protein and cellular DNA denatuation were performed with the addition of P1, P2, and N3 buffers. Clarified supernatant containing plasmid DNA was loaded onto Qiagen spin columns and washed with PB and PE buffers. Plasmid DNA was eluted with 10 mM Tris-HCl buffer.

Transformation procedure: Chemically competent cells were prepared by treatment of exponential phase E. coli cells with ice-cold 50 mM CaCl₂ for 30 min. Competent cells were mixed with plasmid DNA and incubated on ice for 30 min. After 3-5 min heat treatment at 37° C., an equal volume of LB broth was added. Cells were re-grown in a 37° C. incubator for one hours. Transformants were plated on LB agar plates with appropriate antibiotics for plasmid selection. X-gal was included in the plate for the incubation of “endo-blue” indicator strain.

Electroporation procedure: Electro-competent cells were prepared by washing E. coli exponential phase cells in 10% ice-cold glycerol twice (500 ml 10% glycerol for cell pellet from 1 L cell culture). After mixing the DNA with 100 ml of competent cells electroporation was carried out under the condition of 1900 V, 200 W, 25 mF, 0.1 cm cuvette. One ml of LB was added to cells and incubated for 1 hour to amplify the transformants. Transformants were plated on LB agar plates with appropriate antibiotics (Ap, Cm, or Km) for plasmid selection.

Preparation of cell extracts: Cells were cultured overnight in a 37° C. shaker, pelleted by low speed centrifugation (1800 g). Cells were resuspended in a sonication buffer (50 mM Tris-HCl, pH 7.8, 10 mM β-mercaptoethanol, 50 mM NaCl). Cell lysis was completed with sonication at output 4, 50%, discontinuous burst 5 times with a small sonication tip. The lysate was clarified by centrifugation at 14000 g at 4° C. for 10 min. The supernatant (cell extract) was used for the nicking enzyme assay.

DNA sequencing: An AmpliTaq™ dideoxy terminator sequencing kit (Applied Biosystems, Foster City, Calif.)was used in the sequencing reactions. DNA sequences, resolved on an automated sequencer ABI373A, were edited and analyzed using the Seqed program (Applied Biosystems, Foster City, Calif.) and a sequence analysis software package (Accelrys Inc., San Diego, Calif.).

### Example 2

### Cloning of BpmI Restriction-modification System in E. coli

1. Preparation of genomic DNA and restriction digestion of genomic DNA.

Genomic DNA is prepared from Bacillus pumilus (New England Biolabs, Inc. collection #711, Beverly, Mass.) by the standard procedure consisting the following steps:

(a) cell lysis by addition of lysozyme (2 mg/ml final), sucrose (1% final), and 50 mM Tris-HCl, pH 8.0;

(b) cell lysis by addition of 10% SDS (final concentration 0.1%);

(c) cell lysis by addition of 1% Triton X-100 and 62 mM EDTA, 50 mM Tris-HCl, pH 8.0;

(d) phenol-CHCl₃ extraction of DNA 3 times (equal volume) and CHCl₃ extraction one time;

(e) DNA dialysis in 4 liters of TE buffer, change 3×; and

(f) RNA was removed by RNAse A treatment and the genomic DNA was precipitated in ethanol and resupended in TE buffer;

Five μg genomic DNA was digested partially with 2, 1, 0.5, and 0.25 units of ApoI (recognition sequence R/AATTY) at 50° C. for 30 min. Genomic DNA fragments in the range of 2-10 kb were purified through a 1% low-melting agarose gel. Genomic and pBR322 DNA were also digested with AatII, BamHI, ClaI, EagI, EcoRI, HindIII, NdeI, NheI, SalI, and SphI, respectively. Genomic DNA fragments were ligated to pBR322 with compatible ends.

2. Construction of ApoI partial genomic DNA library and challenge of library with BpmI.

The ApoI partial DNA fragments were ligated to EcoRI digested and calf intestinal phosphatase (CIP) (New England Biolabs, Inc., Beverly, Mass.) treated pBR322 vector. The ligated DNA was dialyzed by drop dialysis on 4 L of distilled water and transferred into E. coli RR1 competent cells by electroporation. Ap^{R} transformants were pooled and amplified. Plasmid DNA was prepared from the overnight cells and challenged with BpmI. Following BpmI digestion, the challenged DNA was transformed into RR1 cells. Ap^{R} survivors were screened for resistance to BpmI digestion. A total of 36 plasmid mini-preparations were made. Two resistant clones, #18 and #26, were identified to be resistant to BpmI digestion. AatII, BamHI, ClaI, EagI, EcoRI, HindIII, NdeI, NheI, SalI, and SphI digested genomic DNA were also ligated to pBR322 with compatible ends and genomic DNA libraries were constructed. However, no apparent BpmI resistant clones were discovered from these libraries after screening more than 144 clones.

3. Subcloning and DNA sequencing of the resistant clone.

One resistant clone #26 contains an insert of about 3.1 kb. The forward and reverse primers of pUC19 were used to sequence the insert. Three ApoI and one HindIII fragments were gel-purified and subcloned in pUC19 and sequenced. The rest of the insert was sequenced by primer walking. A methylase gene with high homology to amino-methyltransferase (N6-adenine methylase) was found within the insert which was named BpmIM1 gene. The BpmIM1 gene is 1,650 bp, encoding a 549-amino acid protein with predicted molecular mass of 63,702 daltons.

4. Cloning of BpmI restriction endonuclease gene (BpmIRM) by inverse PCR.

There is one partial open reading frame upstream of BpmIM1 gene that has 31% amino acid sequence identity to another restriction enzyme Eco57I with similar recognition sequence (Eco57I recognition sequence: 5′CTGAAG N16/N14; (Janulaitis et al. Nucl. Acids Res. 20:6051-6056 (1992)); BpmI recognition sequence: 5′CTGGAG N16/N14 (see Rebase). Genomic DNA was digested with restriction enzymes AseI, BclI, HaeII, HpaII, MboI, MseI, NlaIII, PacI, and Tsp509I. The digested DNA was ligated at a low DNA concentration at 2 μg/ml and then used for inverse PCR amplification of BpmIR gene. The sequences of the inverse PCR primers were the following:

| 5′ gtggaaacggaccgtattatggtt 3′ | (SEQ ID NO: 5) |
|---|---|
| (232-34) | |
| 5′ caccagtaaataacaggttattcc 3′ | (SEQ ID NO: 6) |
| (232-35) | |

Inverse PCR conditions were 94° C. 1 min, 55° C. 1 min, 72° C. 2 min for 35 cycles. Inverse PCR products were derived from HaeIII and NlaIII templates, gel-purified from low-melting agarose and sequenced using primers 232-34 and 35.

The primers for the second round of inverse PCR were the following:

| 5′ ttcgtagcaagtacggtccatatcagt 3′ | (SEQ ID NO: 7) |
|---|---|
| (233-76) | |
| 5′ ccgtatgtacttgataggaataacctg 3′ | (SEQ ID NO: 8) |
| (233-77) | |

Genomic DNA was digested with AseI, BclI, BsrFI, BstNI, EcoRI, HincII, HindIII, HpaII, NcoI, PacI, PvuI, TaqI, TfiI, and XbaI. The digested DNA was ligated at a low DNA concentration at 2 μg/ml and then used for inverse PCR amplification of BpmIR gene. Inverse PCR conditions were 94° C. 1 min, 55° C. 1 min, 72° C. 2 min for 35 cycles. Inverse PCR products were derived from AseI, HindIII, HpaII, and TaqI templates, gel-purified from low-melting agarose and sequenced using primers 233-76 and 77.

The primers for the third round of inverse PCR were the following:

| 5′ aggaactaagaaagttcatagctg 3′ | (SEQ ID NO: 9) |
|---|---|
| (234-61) | |
| 5′ atgcggtattatataacccaacag 3′ | (SEQ ID NO: 10) |
| (234-62) | |

Genomic DNA was digested with AflIII, BspHI, BstNI, EcoRI, HaeII, HinP1I, HhaII, HindIII, StyI, and XmnI. The digested DNA was ligated at a low DNA concentration at 2 μg/ml and then used for inverse PCR amplification of BpmIR gene. Inverse PCR conditions were 94° C. 1 min, 55° C. 1 min, 72° C. 2 min for 35 cycles. Inverse PCR products were derived from HinP1I and XmnI templates, gel-purified from low-melting agarose and sequenced using primers 234-61 and 62.

The primers for the fourth round of inverse PCR were the following:

| 5′ tgacgtcctcttcacctaattcgg 3′ | (SEQ ID NO: 11) |
|---|---|
| (235-50) | |
| 5′ gagtttgtgaagatagaaccattg 3′ | (SEQ ID NO: 12) |
| (235-51) | |

Genomic DNA was digested with ApoI, BstBI, BstYI, ClaI, EcoRI, NdeI, RsaI, Sau3AI, SspI, TaqI, and XmnI. The digested DNA was ligated at a low DNA concentration at 2 μg/ml and then used for inverse PCR amplification of BpmIR gene. Inverse PCR conditions were 94° C. 1 min, 55° C. 1 min, 72° C. 2 min for 35 cycles. Inverse PCR products were derived from ApoI, ClaI, NdeI, RsaI, SspI, and TaqI templates, gel-purified from low-melting agarose and sequenced using primers 235-50 and 51. The ClaI fragment (2.4 kb) further extends upstream of BpmIRM gene. The rest of the ClaI fragment was sequenced using primer walking.

After four rounds of inverse PCR reactions, an open reading frame of 3,030 bp was found upstream of BpmI M1 methylase gene, which encodes a 1,009-amino acid protein with predicted molecular mass of 116,891 daltons. This is one of the largest restriction enzymes discovered so far. By amino acid sequence comparison of BpmI endonuclease with all known proteins in GenBank protein database, it was discovered that BpmI endonuclease is a fusion of two distinct elements with possible structural domains of restriction-methylation-specificity (R-M-S). This domain organization is analogous to the type I restriction-modification system with three distinct subunits, restriction, methylation, and specificity (R, M, and S). Because BpmI is distinct from other Type IIs restriction enzymes, it is proposed that BpmI belongs to a subgroup of Type II restriction enzymes called Type IIG.

5. Expression of BpmIM1 gene in E. coli.

Two primers are synthesized to amplify BpmIM1 gene in PCR. The primer sequences are:

| | |
|---|---|
| forward: | |
| 5′ agcggatccggaggtaaataaatgaatcaat | (SEQ ID NO: 13) |
| taattgaaaatgttaat 3′ (238-177) | |
| reverse: | |
| 5′ aagggggcatgcttatacttatttcttcgtt | (SEQ ID NO: 14) |
| ctattgtttct 3′ (238-178) | |

Following digestion with BamHI and SphI, the PCR product was ligated into pACYC184 with the compatible ends. The ligated DNA was transformed into ER2566 competent cells. Cm^{R} transformants were plated at 37° C. overnight. Plasmids with BpmIM1 gene inserts were tested for resistance to BpmI digestion. Two out of 18 clones showed full resistance to BpmI digestion, indicating efficient BpmI M1 expression in E. coli cells and BpmI site modification on the expression plasmid. The host cell ER2566 [pACYC-BpmIM1] was used for expression of BpmIRM gene.

BpmI M1 methylase also modifies XhoI site. XhoI recognition sequence 5′CTCGAG3′ is similar to BpmI recognition sequence 5′CTGGAG3′ with only one base difference. It is concluded that BpmI M1 methylase may recognize the sequence 5′CTNNAG3′ and modify the adenine base to generate N6-adenine in the symmetric recognition sequence.

6. Expression of BpmIRM gene in E. coli using a T7 expression vector.

Two primers were synthesized to amplify the BpmIRM gene. The primer sequences were:

| | |
|---|---|
| 5′ caaggatccggaggtaaataaatgcatataa | (SEQ ID NO: 15) |
| gtgagttagtagataaatac 3′ (247-217) | |
| 5′ ttaggatcctcatttttcttctcctaacgcc | (SEQ ID NO: 16) |
| gctgt 3′ (238-182) | |

The 3,030-bp BpmIRM gene was amplified in PCR using Taq DNA polymerase, digested with BamHI and ligated into BamHI-digested T7 expression vectors pAII17 and pET21a. After transformation of the ligated DNA into ER2566 [pACYC-BpmIM1], Ap^{R} Cm^{R} transformants were screened for the endonuclease gene insert. Seven out of 72 clones contained the insert with correct orientation. However, no BpmI activity was detected in cell extracts of IPTG-induced cells. This was probably due to mutations introduced during the PCR process.

To reduce the mutation frequency, Deep Vent® DNA polymerase was used in PCR reactions to amplify the 3,030-bp BpmIRM gene. The forward primer incorporated an XbaI site and its sequence is the following:

| 5′ caccaatctagaggaggtaaataaatg | (SEQ ID NO: 17) |
|---|---|
| catataagtgagttagtagataaatac 3′ | |
| (238-181) | |

PCR was performed using primers 238-181, 238-182, and Deep Vent® DNA polymerase. The PCR conditions were 94° C. 5 min for one cycle; 94° C. 1 min, 55° C. 1.5 min, 72° C. 8 min for 20 cycles. The PCR product was purified through a Qiagen spin column and digested with BamHI and XbaI and ligated to T7 expression vectors pAII17 and pET21at with compatible ends. Eighteen out of 36 clones contain the correct size insert. Ten ml cell culture for all 18 clones containing inserts were induced with IPTG for 3h and cell extracts were prepared by sonication and assayed for BpmI activity. Clone #4 displayed partial BpmI activity. Because this gene was derived by PCR cloning, the entire BpmIRM fusion gene was sequenced on both strands and it was confirmed to be wild-type sequence.

7. Partial purification of recombinant BpmI activity.

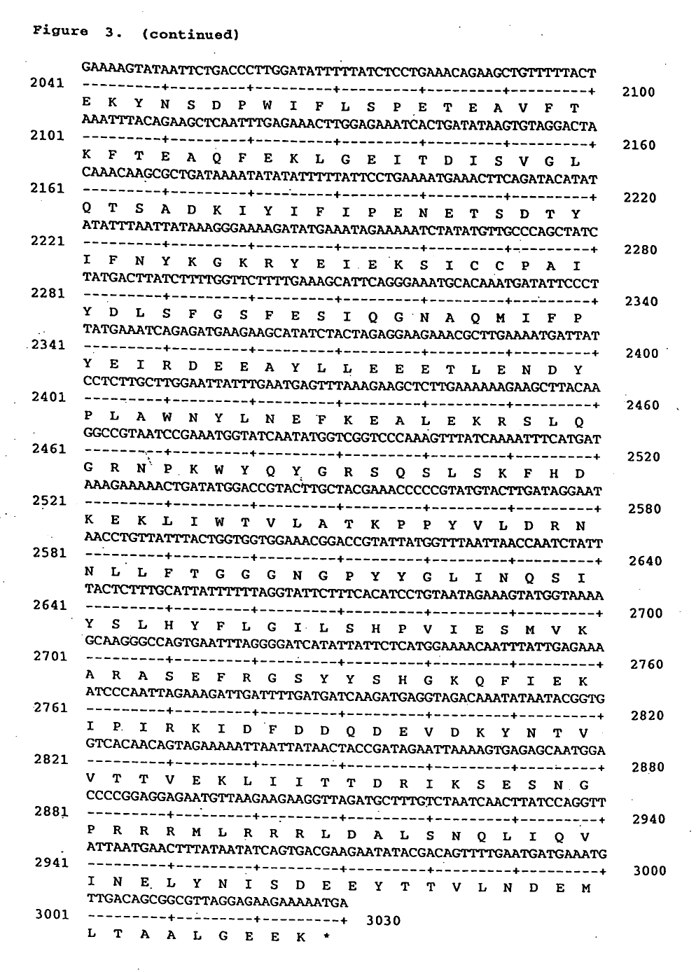

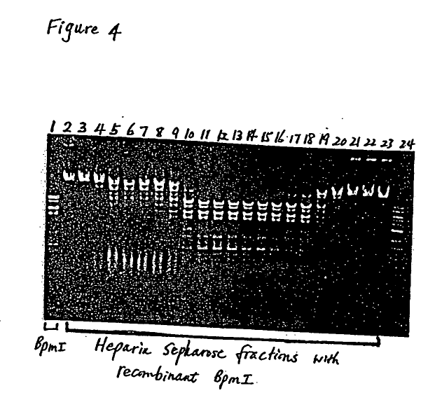

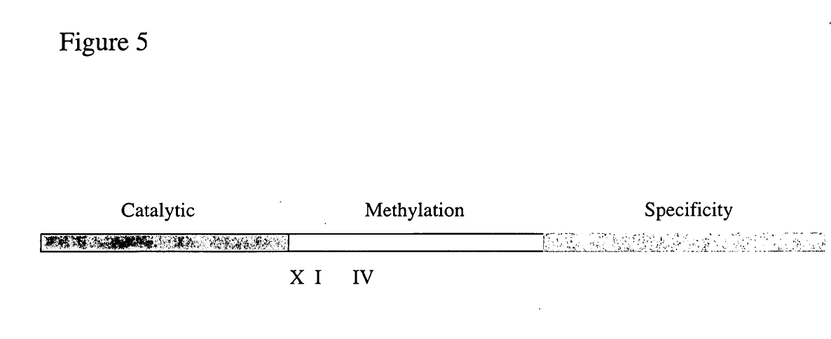

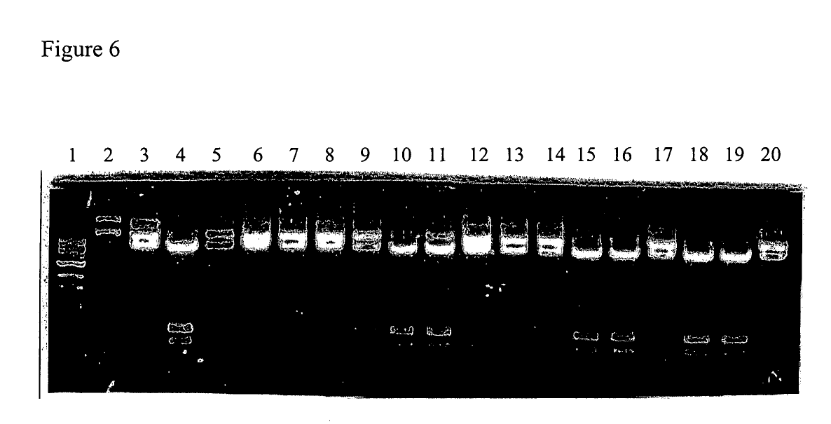

Five hundred ml of cell culture was made for the expression clone #4 ER2566 [pACYC-BpmIM1, pET21at-BpmIRM]. The late log cells were induced with IPTG and cell extract (40 ml) containing BpmI was purified through a heparin Sepharose column. Proteins were eluted with a NaCl gradient of 50 mM to 1 M. Fractions 6 to 27 contained the most protein concentration and were assayed for BpmI activity on λ DNA. It was found that fractions 15 to 18 contained the most active BpmI activity (FIG. 4). The yield was estimated at 1,800 units of BpmI per gram of wet E. coli cells. The specific activity was estimated at 24,000 units per mg of protein. Proteins from fractions 15 to 18 were analyzed on a SDS-PAGE gel and protein bands were stained with Gelcode blue stain. A protein band corresponding to ˜115 kDa was detected on the protein gel, in close agreement with the predicted size of 117 kDa.

The E. coli strain ER2566 [pACYC-BpmIM1, pET21at-BpmIRM] has been deposited under the terms and conditions of the Budapest Treaty with the American Type Culture Collection on Jan. 22, 2001 and received Accession No. PTA-2598.

### Example 3

### Deletion of the Methylase Portion of BpmI RM Fusion Protein

Two primers were synthesized to amplify the putative endonuclease domain with deletion of the methylase and specificity domains. The deletion clone thus contains only the R portion and the M and S portions were removed. The forward primer was 238-181 as described above. The reverse primer had the following sequence with a XhoI site at the 5′ end:

| | |
|---|---|
| 5′ tgaaatctcgagttatcctgatccacaaca | (SEQ ID NO: 18) |
| tatatctgctat 3′ (244-95) | |

The deletion junction was in motif I of γ type N6 adenine methylase. The γ type N6 adenine methylases contain conserved motifs of X, I, II, III, IV, V, VI, VII, VIII. The specificity domain (TRD) is located after motif VIII. The BpmI deletion clone (BpmI-Δ#1) still carried motifs X and part of motif I. The specificity domain after motif VIII was also deleted (the remaining portion is shown in FIG. 1).

PCR was performed using primers 238-181 and 244-95 and Taq plus Vent® DNA polymerase (94° C. 1 min, 60° C. 1 min, and 72° C. 1 min for 25 cycles). The PCR product was digested with XbaI and XhoI and cloned into a T7 expression vector pET21b. Sixteen clones out of 36 screened contained the correct size insert and the cells were induced with IPTG for 3h. Cell extract was prepared by sonication and assayed for BpmI activity on λ DNA. However, no apparent BpmI digestion pattern was detected. Only non-specific nuclease was detected in cell extract, resulting in a smearing of DNA substrate. It was concluded that deletion of the methylase and specificity portion of the BpmIRM fusion protein abolished BpmI restriction activity.

To further confirm the above result, another deletion clone was constructed that deleted methylase motifs IV, V, VI, VII, VIII, and the specificity domain. This EcoRI fragment deletion mutant contains 1,521 bp (507 amino acid) deletion at the C-terminus half of the fusion protein (BpmI-Δ#2). IPTG-induced cell extract of this mutant also did not display BpmI endonuclease activity.

To delete the specificity domain referred to as the target-recognizing domain (TRD), a HindIII fragment of 579 bp (193 amino acid) was deleted from the C-terminus of BpmI RM fusion endonuclease (BpmI-Δ#3). IPTG-induced cell extract of the TRD deletion mutant did not show any BpmI endonuclease activity. However, the mutant protein displayed non-specific nuclease activity. It was concluded that the TRD is also required for BpmI endonuclease activity. Deletion of the TRD may abolish or reduce its DNA binding affinity and specificity. By swapping in other N6 methylase and specificity domains, new enzyme specificities can be created.

### Example 4

### Generation of New Enzyme Specificity Using BpmI RM Fusion Protein

Since BpmI endonuclease consists of three domains (R-M-S), it is possible to plug in other methylation-specificity domains to create a new enzyme specificity. The BpmIRM fusion gene is cloned in a T7 expression vector as described in Example 1. Plasmid DNA is prepared. The γ type N6 adenine methylases contain conserved motifs of X, I, II, III, IV, V, VI, VII, VIII (Malone T. et al. J.Mol.Biol 253:618-632 (1995)). Motifs X through VIII and TRD are deleted and a DNA linker coding for one, 3, 5, 7 and 10 bridging amino acids are inserted with a restriction site, preferably a blunt end restriction site, for example, the SmaI site. The length of the DNA linker is sufficient to provide steric space for the introduction of the new M-S domains. DNA coding for other γ type N6 adenine methylases containing motifs of X, I, II, III, IV, V, VI, VII, VIII and TRD are ligated to the digested blunt site (in frame) of the BpmI deletion clone. The ligated DNA is transformed into a non-T7 expression vector. After the insert is verified, the plasmid containing new methylation-specificity domains is transformed into a T7 expression host and induced with IPTG. Cell extract is assayed on plasmid and phage DNA and analyzed for new restriction activity.

### Example 5

### Construction of a Cleavage Deficient Variant of BpmI

Modification of the specificity of BpmI, AcuI, and BsgI which are all Type IIG restriction enzymes that display both endonuclease and methyltransferase (R-M) activity is achieved as follows:

Optional mutation of the restriction endonuclease was carried out to increase viability of cells transformed with an enzyme having altered specificity.

A two-step PCR mutagenesis was carried out to mutate the Asp74 codon (Asp74 to Ala74) in the catalytic domain. The PCR primers have the following sequences:

| | |
|---|---|
| PCR reaction 1: | |
| Forward primer (P1): | |
| 5′ CACCAATCTAGAGGAGGTAAATAAATGCATA | (SEQ ID NO: 19) |
| TAAGTGAGTTAGTAGATA AATAG 3′ | |
| (TCTAGA, XbaI site) | |
| Reverse mutagenic primer (P2): | |
| 5′ GTTTATACGAAGTGTATAAGCTGGATTTTTC | (SEQ ID NO: 20) |
| TTTGTCTG 3′ | |
| PCR reaction 2: | |
| Forward mutagenic primer (P3): | |
| 5′ GAGACAAAGAAAAATCCAGCTTATACACTTC | (SEQ ID NO: 21) |
| GTATAAAC 3′ | |
| Reverse primer (P4): | |
| 5′ TTAGGATCCTCATTTTTCTTCTCCTAACGCC | (SEQ ID NO: 22) |
| GCTGT 3′ (GGATCC, BamHI site) | |

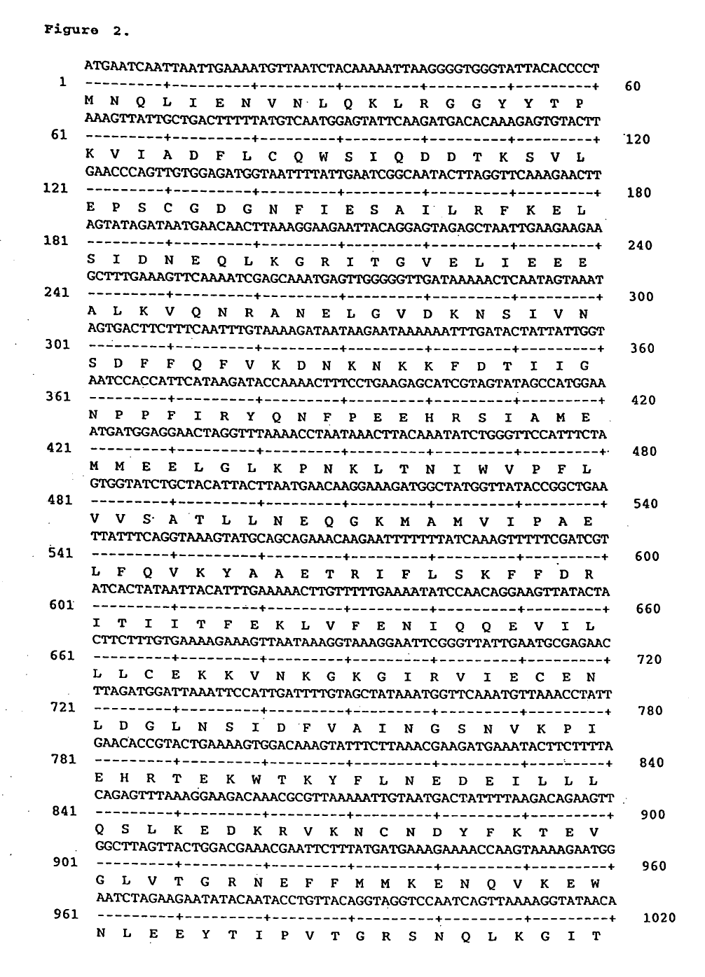

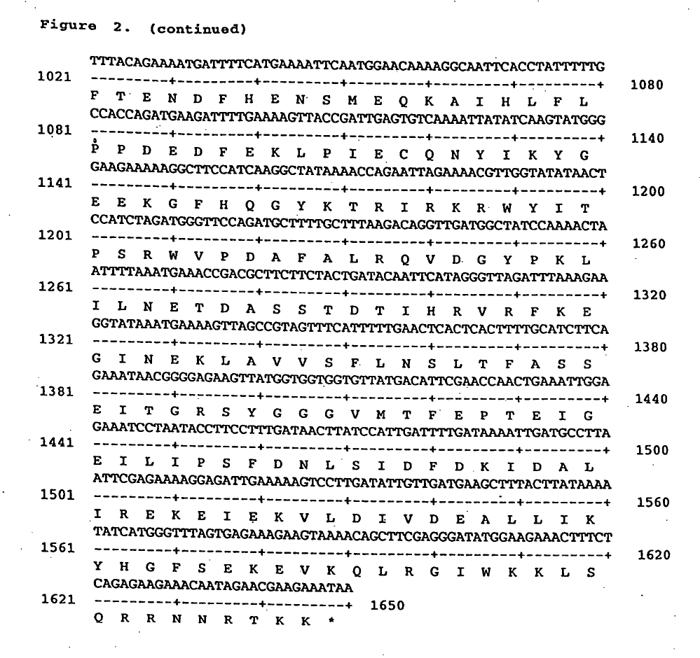

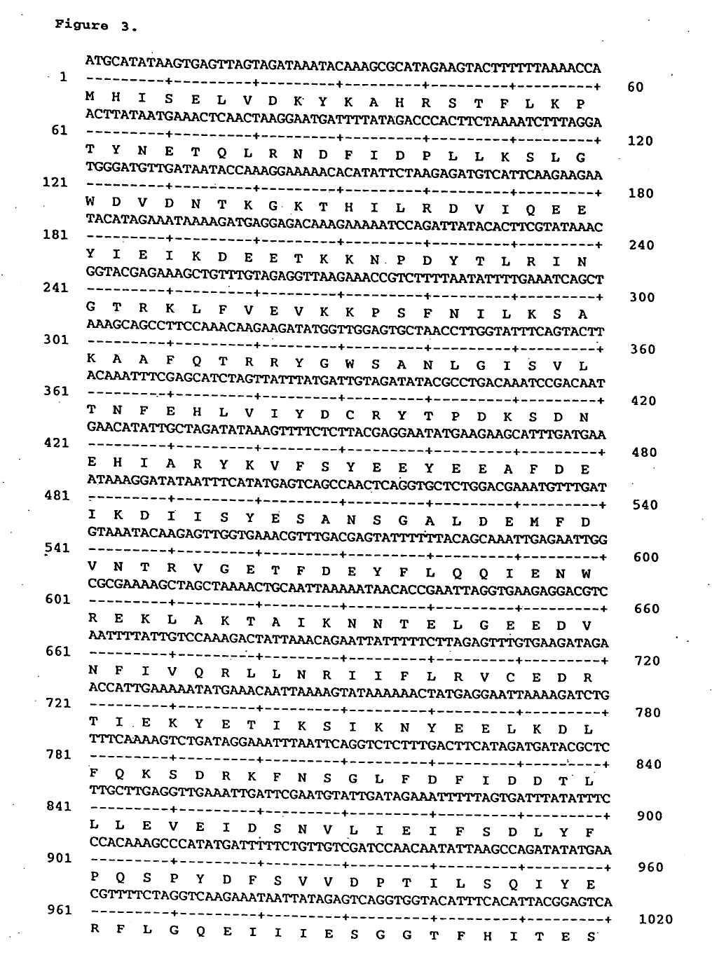

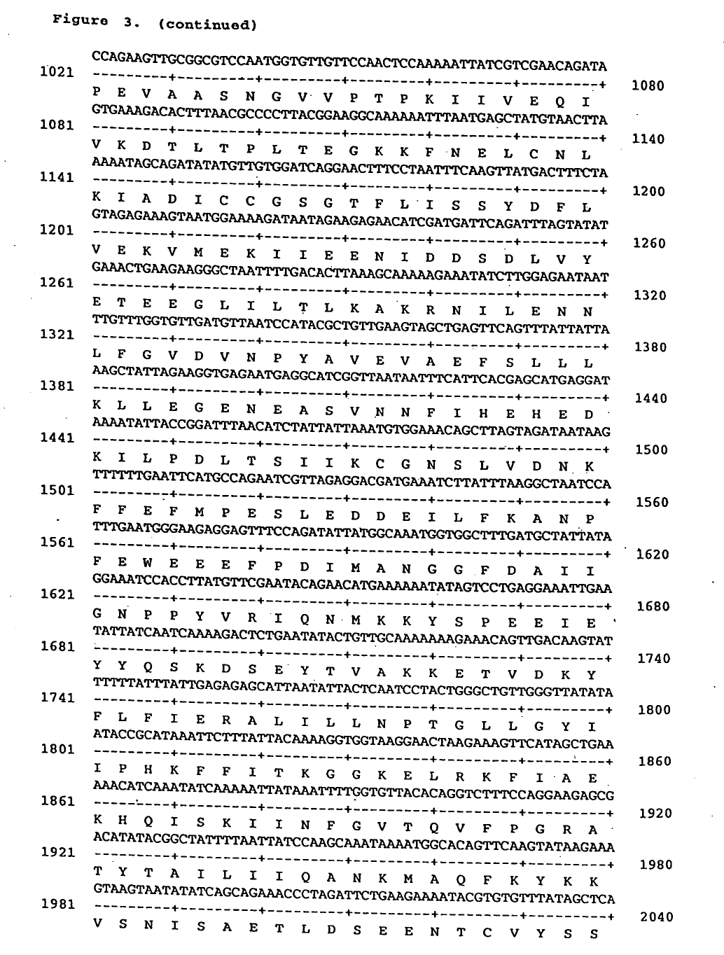

The N-terminal 300 bp coding sequence was amplified in PCR reaction 1 with the following PCR conditions: 94° C. for 5 min, 1 cycle; 94° C. for 30 sec, 55° C. for 30 sec, 72° C. for 30 sec for 20 cycles; 72° C., 7 min for 1 cycle, 4 units of Vent® DNA polymerase. The rest of the coding sequence was amplified in PCR reaction 2 with the following PCR condition: 94° C. for 5 min, 1 cycle; 94° C. for 30 sec, 55° C. for 30 sec, 72° C. for 1 min for 20 cycles; 72° C., 7 min for 1 cycle, 4 units of Vent® DNA polymerase. PCR products 1 and 2 were purified from a low-melting agarose gel and used as the template for PCR assembly using primers P1 and P4. The assembly PCR conditions were 94° C. for 5 min, 1 cycle; 94° C. for 30 sec, 55° C. for 30 sec, 72° C. for 3 min for 20 cycles; 72° C., 7 min for 1 cycle. The mutagenized PCR product was purified and digested with XbaI and BamHI and cloned into a T7 expression vector pET28a. The phenotype of the resulting BpmI variant should be R⁻ (cleavage deficient) and M⁺ (methylation proficient). After screening 18 plasmids for PCR insert and digestion with BpmI endonuclease, 11 clones were found to be resistant to BpmI digestion (data shown in FIG. 2). Endonuclease activity was not detected in any of the mutant extracts prepared from IPTG-induced cells. Because multiple rounds of PCR were performed to generate the R⁻M⁺ variant D74A, it was necessary to re-sequence the entire gene to confirm that no other mutations were introduced. Six sequencing primers were used to sequence the entire gene. R⁻M⁺ variant D74A clone #4 carried one additional amino acid change at E1007G. In a separate experiment, it was determined that E1007G substitution was not important to BpmI endonuclease activity. The Asp74 to Ala74 substitution abolished BpmI endonuclease activity.

Using the same PCR mutagenesis strategy, another BpmI R⁻M⁺ variant E88A was constructed. It was confirmed that E88A is deficient in endonuclease activity and proficient in methylase activity. Both D74A and E88A mutants are useful as the recipient for exchange of a new specificity domain to generate novel enzymes. After a new specificity is confirmed by DNA binding assays, the mutated residues D74A or E88A are changed back to the wild-type residue Asp74 or Glu88 to restore the endonuclease activity. A non-cognate methylase can be used to protect the host DNA.

### Example 6

### Construction of a Deletion Mutant Carrying Deletions in the Methylase Domain and Specificity Domain

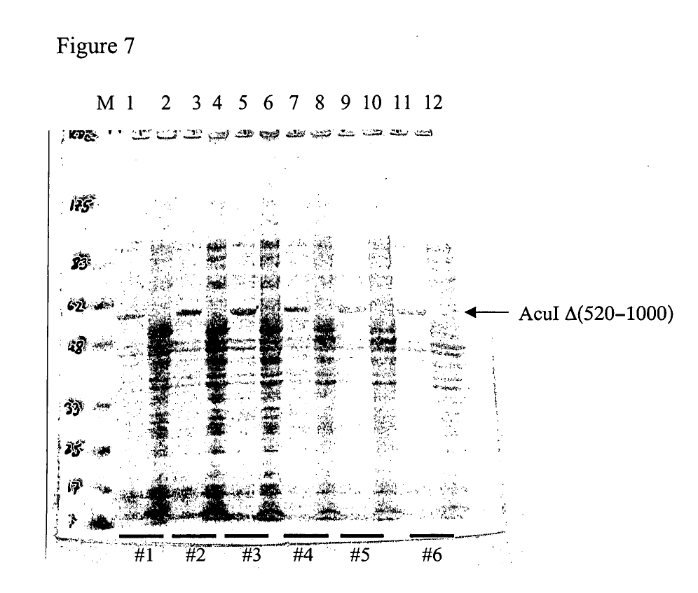

The methylase domains of BpmI and AcuI belong to the g type N6 adenine methylases. Motif IV is a conserved methylase block and has a GNPPY sequence in both of the BpmI and AcuI methylase domains. This site was chosen as a fusion junction for making chimeric enzymes. An AcuI deletion mutant was constructed that deleted methylase motif IV and the remaining C-terminal coding sequence. The starting AcuI enzyme was a cleavage-deficient variant D80A (R-M+mutant). The codon Phe520 was mutated to a stop codon by PCR mutagenesis to generate variant AcuI D80A/Δ(520-1000). The deletion mutant protein was expressed in E. coli ER2566 via T7 expression vector pET28a. When the cells were induced with IPTG (3 hours induction at 37° C.), a prominent protein band of 59 kDa was detected in SDS-PAGE gel (data shown in FIG. 7). The deletion mutant AcuI D80A/Δ (520-1000) is soluble in E. coli cell extract and not degraded by E. coli proteases. This deletion mutant can be used as the backbone to construct chimeric Type IIG enzymes. DNA coding for similar methylase motifs IV to VIII and an alternate specificity determinant can be ligated to this deletion mutant to construct a functional chimeric enzyme.

The coding sequences are ligated together by T4 DNA ligase (blunt end or sticky end ligation). In an alternative embodiment, the two coding sequences are assembled together by a two-step PCR method as described in Example 7. After the new specificity is determined by DNA binding assays, the catalytic residue Asp80 is restored. Although not required always, a non-cognate methylase is used here for protection of the host cell after transformation with the fusion DNA encoding the chimeric protein.

### Example 7

### Expression of a Natural Deletion Mutant of ThaIVp, a Truncated Type IIG Enzyme

ThaIVp is derived from a thermophilic bacterium. The ThaIVp coding sequence was amplified from the genomic DNA of Thermoplasma acidophilum (ATCC #25905) by PCR. The PCR primers have the following sequences:

| Forward: | |
|---|---|
| 5′ GGTGGTTCTAGAGGAGGTAAATAAATGTCTA | (SEQ ID NO: 23) |
| ATGAAAATTATAACATTGATTTC 3′ | |
| (TCTAGA, XbaI site) (293-282) | |
| Reverse: | |
| 5′ GGTGGTGAGCTCCTATTGACATAATCGATCA | (SEQ ID NO: 24) |
| TCAAGAAG 3′ (GAGCTC SacI site) | |
| (293-283) | |

The PCR components are as following: 293-282, 293-283 (0.8 mM), 4 units of Vent® DNA polymerase, Thermoplasma acidophilum (ATCC # 25905) genomic DNA (1 mg), dNTP (4 mM), 1x thermophilic polymerase buffer, H₂O73 μl, and MgSO₄ at 2, 4, and 6 mM. The PCR conditions are 95° C. 5 min for 1 cycle; followed by 95° C. 30 sec, 57° C. 30 sec, 72° C. 3 min for 30 cycles. The PCR product was digested by XbaI and SacI and ligated to pET-28a with compatible ends and transferred to T7 expression host ER2566 by transformation. There is a natural stop codon at the end of ThaIVp before the conserved methylase motif IV. Therefore the natural deletion mutant of ThaIVp is used as the recipient backbone for generating chimeric Type IIG enzymes by addition (ligation) of motif IV and the remaining methylase motifs and the specificity domain. This DNA segment can be ligated to DNA encoding thermophilic methylase domains (IV to X) and a specificity domain to produce a thermostable chimeric enzyme.

### Example 8

### Construction of a Chimeric Enzyme Between BpmI and BsgI

The DNA recognition sequences for BpmI and BsgI are CTGGAG and GTGCAG, respectively. BsgI endonuclease is a Type IIG enzyme that shares 35.4% amino acid sequence identity to BpmI. A chimeric enzyme was constructed between BpmI and BsgI, in which the N-terminal coding sequence (catalytic domain plus methylase motifs I to III) was derived from BpmI and the C-terminal coding sequence (methylase motifs IV to X and the specificity domain) was derived from BsgI. The chimeric coding sequence was generated by a two-step PCR reaction. PCR primers were designed that can anneal to methylase motif IV on both BpmI and BsgI templates. The amino acid sequences in the fusion junction are shown below:

| | |
|---|---|
| BpmI | F D A I I G N P P Y |
| BsgI | F D V I L G N P P Y |

The forward primer P1 described in Example 5 and a new reverse primer P2′ were used to amplify the N-terminal coding sequence from BpmIRM gene.

The new reverse mutagenic primer P2′ has the following sequence:

| | |
|---|---|
| 5′ ATAGGGTGGATTGCCTAATATTACATCAAAG | (SEQ ID NO: 25) |
| CCACCATTTGC 3′ (P2′). | |

PCR conditions were 94° C. for 5 min, 1 cycle; 94° C. for 30 sec, 55° C. for 30 sec, 72° C. for 2 min for 17-22 cycles; 72° C., 7 min for 1 cycle with 4 units of Vent® DNA polymerase.

The forward mutagenic primer in the fusion junction has the following sequence:

| | |
|---|---|
| 5′ TTTGATGTAATATTAGGCAATCCACCCTATA | (SEQ ID NO: 26) |
| TAAGAATTC 3′ (P3′) | |

Since the BsgIRM gene was cloned in pUC19, primer P3′ and a pUC universal primer NEB #1221 was used to amplify the C-terminal BsgI coding sequence. PCR conditions were 94° C. for 5 min, 1 cycle; 94° C. for 30 sec, 55° C. for 30 sec, 72° C. for 2 min for 15-22 cycles; 72° C., 7 min for 1 cycle with 4 units of Vent® DNA polymerase. The PCR products were purified from a low-melting agarose gel and assembled by PCR using primers P1′ and pUC universal primer #1221 (New England Biolabs, Inc., Beverly, Mass.). The PCR conditions were 94° C. for 5 min, 1 cycle; 94° C. for 30 sec, 55° C. for 30 sec, 72° C. for 3 min 10 sec for 15 cycles; 72° C., 7 min for 1 cycle with 4 units of Vent® DNA polymerase. The PCR DNA fragment was cloned into pET21at and transformed into T7 expression host ER2566. E. coli host with pACYC-BpmIM or pACYC-BsgIM was also used for transformation. The fusion junction was confirmed by DNA sequencing.

E. coli strain ER1992 carries the dinD::IacZ fusion (the dinD DNA damage inducible promoter is fused to the lacZ gene). When bacterial DNA is damaged by double-stranded cuts or single-stranded nicks, UV radiation, or interference with DNA replication, the indicator strain forms blue colony on X-gal plates. When plasmids carrying the chimeric BpmI/BsgI R-M fusion were transformed into the endo-blue indicator strain ER1992 (dinD::IacZ), they caused formation of blue colonies in the absence of IPTG induction. This indicates transformants suffered DNA damage resulting from constitutive expression of the fusion protein (data shown in FIG. 4). The transformants initially formed blue colonies on X-gal plates. When these cells were plated on X-gal plates with IPTG, most cells turn white. After IPTG induction, cells suffered lethal level of DNA damage and died. The cells carrying inactive mutant version of the chimeric R-M fusion protein took over the population and formed the white colonies. When the blue transformants were re-streaked on X-gal plates, about two-third remains blue colonies, and one-third form white or partial blue colonies.

What is claimed is:

1. A method for altering the cleavage specificity of a Type IIG restriction endonuclease, the Type IIG restriction endonuclease characterized by a cleavage domain adjacent to a methylase domain, the methylase domain located adjacent to a specificity domain, the method comprising:
(a) ligating a first DNA sequence and a second DNA sequence to form a fusion DNA , wherein
(i) the first DNA sequence comprises a DNA segment encoding a catalytic domain and an N-terminal portion of a methylase domain of a first Type IIG restriction endonuclease, and
(ii) the second DNA sequence, comprises a DNA segment encoding a specificity domain and a C-terminal portion of a methylase domain of a second Type IIG restriction endonuclease;
such that the ligation occurs between sequences encoding the methylase domain; and
(b) transforming a host cell with the fusion DNA for expressing a Type IIG restriction endonuclease with altered cleavage specificity.

2. A method according to claim 1, wherein step (a) further comprises: introducing a mutation into the cleavage domain to enhance the viability of the transformed host cell.

3. A method according to claim 1, wherein the sequence corresponding to the N-terminal portion of the methylase terminates in a methylase conserved motif selected from motifs X, I, II, III, IV, V, VI, VII or VIII.

4. A method according to claim 1, wherein the sequence corresponding to the C-terminal portion of the methylase terminates in a methylase conserved motif.

5. A method according to claims 3 or 4 wherein the conserved motif is selected from motifs X, I, II, III, IV, V, VI, VII or VIII wherein the N-terminal portion and the C-terminal portion of the methylase are non-overlapping.

6. A method according to claims 3 or 5, wherein the sequence corresponding to the N-terminal portion of the methylase motif terminates between the sequence encoding motif III and NPPY in motif IV.

7. A method according to claim 1, wherein ligation occurs by means of a linker sequence attached to each of the N-terminal portion of the methylase domain and the C-terminal portion of the methylase domain on the first and second DNA segment.

8. A method according to claim 1, wherein the fusion DNA encodes an active methylase domain.

9. A method according to claim 1, wherein the first and second Type IIG endonucleases have defined cleavage and recognition sites.

10. A method according to claim 1, wherein the first Type IIG endonuclease has a defined cleavage and recognition site and the second Type IIG endonuclease is characterized by a bioinformatic search of a microbial sequence database.

11. A method for forming a non-natural, functional Type IIG restriction endonuclease, wherein the Type IIG restriction endonuclease is characterized by a functional cleavage domain, a functional methylase domain and an altered functional specificity domain, compared with a natural form of the functional Type IIG endonuclease, comprising:
(a) inserting into a DNA encoding the methylase domain or the specificity domain of the natural form of the functional Type IIG endonuclease, a mutation or a nucleic acid linker sequence for inactivating optionally the cleavage domain and inactivating (i) the functional methylase domain and the specificity domain or (ii) the functional methylase domain or the functional specificity domain;
(b) ligating to the DNA at the mutation or at the linker, a DNA encoding (i) a portion of the methylase and specificity domain or (ii) a portion of the methylase or specificity domain to form a fusion DNA; and
(c) transforming a host cell having a marker for detecting a colony expressing a non-natural functional Type IIG restriction endonuclease.

12. A method according to claim 11, wherein the mutation is positioned within a conserved motif in the methylase domain.

13. A method according to claim 11, wherein the mutation is a deletion at a 5′-end of the DNA encoding the specificity domain.

14. A method according to claim 11, wherein the mutation is a deletion within the specificity domain.

15. A method according to claim 11, wherein the linker is a transposon mediated linker insertion sequence.

16. A method according to claim 11, wherein the linker contains a restriction endonuclease cleavage site which is unique within the DNA encoding the restriction endonuclease.
